(19) United States
(12) Patent Application Publication
(10) Pub. No.: US 2024/0148559 A1
(43) Pub. Date: May 9, 2024

(54) WOUND DRESSING AND METHOD OF MANUFACTURE

(71) Applicant: Smith & Nephew PLC, Watford (GB)

(72) Inventor: Amy Nicole Wheldrake, Goole (GB)

(21) Appl. No.: 18/388,484

(22) Filed: Nov. 9, 2023

Related U.S. Application Data
(63) Continuation of application No. 16/634,842, filed on Jan. 28, 2020.

(30) Foreign Application Priority Data
Jul. 28, 2017 (GB) 1712165.8

(51) Int. Cl. A61F 13/05; A61F 13/01; A61F 13/0206; D04H 1/4382; D04H 1/4391; D04H 1/541

(52) U.S. Cl. CPC A61F 13/05; A61F 13/01029; A61F 13/0209; D04H 1/43828; D04H 1/4383; D04H 1/43835; D04H 1/43838; D04H 1/43912; D04H 1/5412; D04H 1/5416; D04H 1/5418

(57) ABSTRACT

A wound product and a method of manufacturing the wound product is provided. The wound product comprises a first layer comprising a plurality of splittable fibres. At least some of the plurality of splittable fibres are split longitudinally along at least part of their length, and at least some of the plurality of splittable fibres are entangled.

The present invention relates to a wound product and a method of manufacturing such a wound product. In particular, but not exclusively, embodiments of the present invention relate to the method of manufacturing a wound product including a plurality of splittable fibres, at least some of which are split longitudinally along at least part of their length, which provides greater absorbency.

### BACKGROUND

In wound products such as wound dressings, it is desirable to have high absorbency. When engineering nonwovens for high absorbency, it is an aim to achieve as greater fibre surface area as possible. To achieve this fibres with a small diameter are used. A small diameter equates to a smaller linear density—the measure of this in fibres is decitex (dtex). Using fibres with a small diameter in the wound dressing allow a greater fibre surface area to be achieved.

Current nonwovens manufacturing processes are capable of working with fibres around 3 dtex. However, for fibres having lower dtex such processing becomes problematic. Accordingly, for such processes there is an effective lower limit to the linear density (estimated to be around 1 dtex) in the fibres that can be worked with for producing nonwovens, and so a corresponding limit in the fibre surface area and thus absorbency in such nonwovens.

It would therefore be beneficial to provide a wound product which includes fibres of even smaller diameter, thereby increasing fibre surface area within the wound product and so improving absorbency.

### SUMMARY

According to a first aspect of the present invention there is provided a method of manufacturing a wound product, the method comprising: forming a first layer comprising a plurality of splittable fibres; and processing the first layer such that at least some of the plurality of splittable fibres are split longitudinally along at least part of their length; wherein the processing results in mechanical entanglement of the splittable fibres.

According to a second aspect of the present invention there is provided a method of manufacturing a wound product, the method comprising: forming a first layer comprising a plurality of splittable fibres; processing the first layer such that at least some of the plurality of splittable fibres are split longitudinally along at least part of their length; and subsequently performing a process that results in mechanical entanglement of the splittable fibres.

According to a third aspect of the present invention there is provided a method of manufacturing a wound product, the method comprising: forming a first layer comprising a plurality of splittable fibres; performing a process that results in mechanical entanglement of the splittable fibres; and subsequently processing the first layer such that at least some of the plurality of splittable fibres are split longitudinally along at least part of their length.

According to a fourth aspect of the present invention there is provided a wound product obtained by the method of the first, second or third aspect of the present invention.

According to a fifth aspect of the present invention, there is provided a wound product comprising a first layer, the first layer comprising a plurality of splittable fibres; wherein at least some of the plurality of splittable fibres are split longitudinally along at least part of their length; and wherein at least some of the plurality of splittable fibres are entangled.

According to a sixth aspect of the present invention, a method of operating a negative pressure wound system is provided. The method comprising: operating a negative pressure source fluidically connected to a wound product according to the fourth aspect or the fifth aspect of the present invention, the wound product configured to be positioned over a wound.

According to a sixth aspect of the present invention, there is provided a negative pressure wound therapy kit comprising a wound product according to the fourth or fifth aspect of the present invention and a negative pressure source configured to be fluidically connected to the wound product.

According to a seventh aspect of the present invention, there is provided a method of treating a wound using a wound product according to the fourth aspect or the fifth aspect of the present invention.

According to an eighth aspect of the present invention, there is provided a method of providing negative pressure wound therapy to a wound, the method comprising: placing a wound product according to the fourth or fifth aspect of the present invention over a wound; forming a fluid flow path between the wound product and a negative pressure source; and operating the negative pressure source to provide negative pressure to the wound.

Certain embodiments of the present invention provide the advantage that a wound product with greater surface area is provided.

Certain embodiments of the present invention provide the advantage that a wound product having greater absorbency is provided.

Certain embodiments of the present invention provide the advantage of an increase in moisture vapour transmission rates.

Certain embodiments of the present invention provide the advantage of improved fluid handling.

### BRIEF DESCRIPTION OF THE DRAWINGS

Embodiments of the invention are further described hereinafter with reference to the accompanying drawings, in which:

FIG. 1 illustrates a flow of a method of manufacturing an example of a wound product;

FIG. 2A illustrates an example of a splittable fibre structure;

FIG. 2D illustrates another example of a splittable fibre structure;

FIG. 2C illustrates another example of a splittable fibre structure;

FIG. 2D illustrates another example of a splittable fibre structure;

FIG. 2E illustrates another example of a splittable fibre structure;

FIG. 3 illustrates an example of a splittable fibre before and after splitting;

FIG. 4A is a schematic diagram of an example of a wound product;

FIG. 4B is a schematic diagram of another example of a wound product;

FIG. 5 illustrates an example of an apparatus for manufacturing an example of a wound product; and

FIG. 6 is a schematic diagram of an example of a negative pressure wound therapy system using an example of a wound product.

In the drawings like reference numerals refer to like parts.

### DETAILED DESCRIPTION

As used herein the expression “wound” may include an injury to living tissue may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound becomes a chronic wound when it does not follow the healing stages resulting in a lengthened recovery. It is believed that the transition from acute to chronic wound can be due to a patient being immuno-compromised.

Chronic wounds may include for example: Venous ulcers: Venous ulcers usually occur in the legs, account for the majority of chronic wounds, and mostly affect the elderly, Diabetic ulcers (typically foot or ankle ulcers, Peripheral Arterial Disease, Pressure ulcers, or Epidermolysis Bullosa (EB).

The wound may also include a deep tissue injury. The deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

Treatment of wounds such as at least one of those described above can be performed using negative pressure wound therapy, wherein a reduced or negative pressure can be applied to the wound to facilitate and promote healing of the wound. It will also be appreciated that the wound product and methods as disclosed herein may be applied to other parts of the body, and are not necessarily limited to treatment of wounds.

It will be understood that certain embodiments of the present disclosure are generally applicable to use in topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (e.g., −40 mmHg is less than −60 mmHg). Negative pressure that is “more” or “greater” than −X mmHg corresponds to pressure that is further from atmospheric pressure (e.g., −80 mmHg is more than −60 mmHg). In some embodiments, local ambient atmospheric pressure is used as a reference point, and such local atmospheric pressure may not necessarily be, for example, 760 mmHg.

The negative pressure range for certain embodiments of the present disclosure can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In certain embodiments, the pressure range can be between about −40 mmHg and −150 mmHg. Alternatively a pressure range of up to −75 mmHg, up to −80 mmHg or over −80 mmHg can be used. Also in other embodiments a pressure range of below −75 mmHg can be used. Alternatively, a pressure range of over approximately −100 mmHg, or even −150 mmHg, can be supplied by the negative pressure apparatus.

In certain embodiments, increased wound contraction can lead to increased tissue expansion in the surrounding wound tissue. This effect may be increased by varying the force applied to the tissue, for example by varying the negative pressure applied to the wound over time, possibly in conjunction with increased tensile forces applied to the wound. In certain embodiments, negative pressure may be varied over time for example using a sinusoidal wave, square wave, or in synchronization with one or more patient physiological indices (e.g., heartbeat).

According to one definition, a microfibre is a fibre (including staple fibres and filaments) of linear density approximately 1 decitex (dtex) or less. The term microfibre may also be limited to fibres of linear density above 0.3 dtex, where fibres of 0.3 dtex or less are then referred to as super-microfibres. In a similar manner, when a super-microfibre has a cross-sectional dimension within a range or on the order of nanometres (for example, fibres of <0.1 dtex), it may be referred to as a nanofibre.

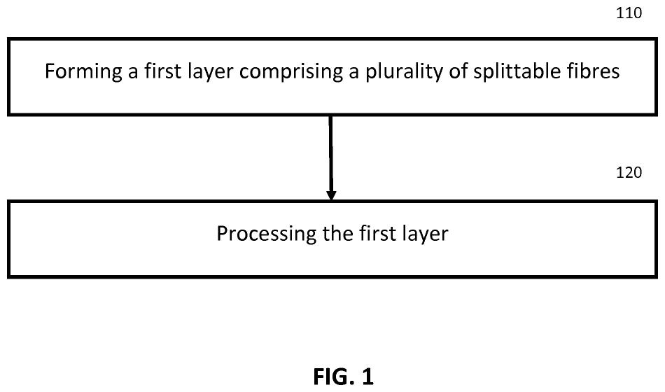

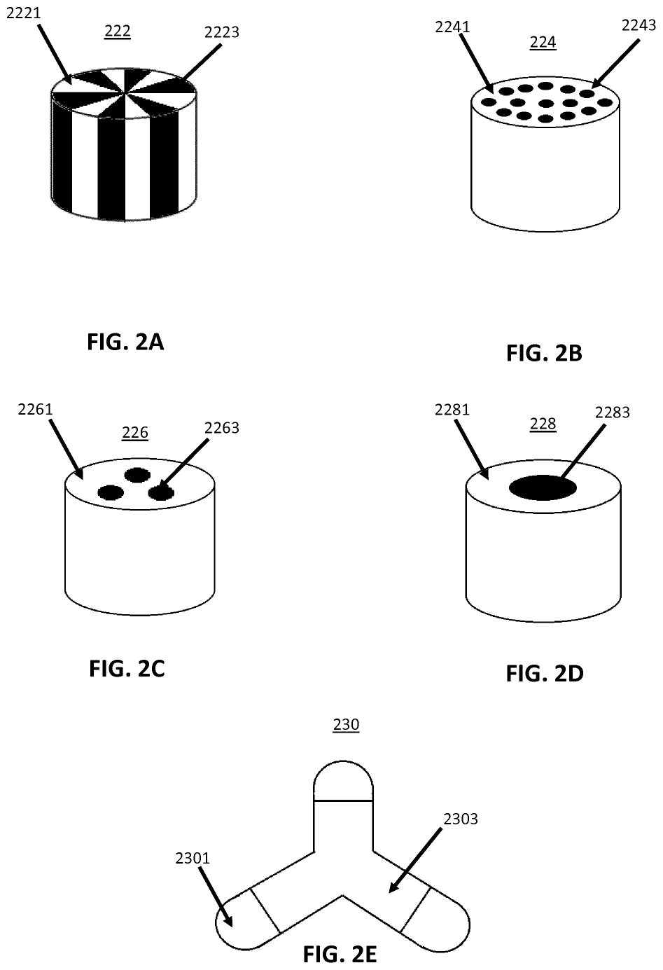

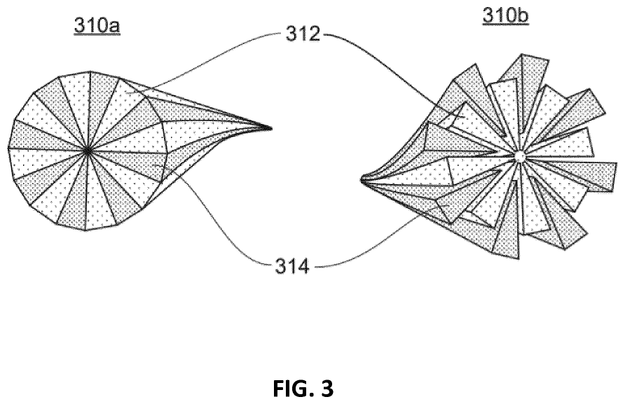

FIG. 1 illustrates an exemplary method of manufacturing a wound product which includes a plurality of splittable fibres. Examples of splittable fibres are illustrated in FIG. 2, and an example of a splittable fibre before and after processing is shown in FIG. 3.

In the following a wound product will be referred to in general terms. However, in certain embodiments of the present invention, the wound product is a wound dressing. Furthermore, in certain other embodiments of the present invention, the wound product is a wound filler. It will be appreciated that the wound product of the present invention may therefore be used as a wound dressing or a wound filler (where these uses should be considered as non-limiting), where certain embodiments for each use may provide further modifications specific to each particular use. For example, in general a wound filler may relate to a wound product which is at least partly put into a wound, and a wound dressing may relate to a wound product which is at least partly put over or placed onto a wound. Additionally, in other embodiments, the wound product may be a debrider or a wound cleaner.

In step 210, the method includes forming a first layer including a plurality of splittable fibres. In certain embodiments, a layer of splittable fibres in this pre-processed state may be referred to as a splittable fibre web. In certain embodiments of the present invention, a splittable fibre web may be formed by an air laid process, carding or a wet-laid process.

In certain embodiments of the present invention, a splittable fibre may be a bicomponent fibre that may be split into constituent fibres (that is, sub-fibres), along at least part of its length, during processing. Such processing may be when processing the fibre into a nonwoven fabric. Such splitting may be in a longitudinal direction of the fibre. It will be appreciated that a splittable fibre of the present invention is not limited to a bicomponent fibre, and indeed may be a splittable fibre which includes more than two constituent fibres wherein at least one of the constituent fibres may be separated from another constituent fibre thereby splitting the splittable fibre as described above.

After such splitting, a section of a splittable fibre may still be whole, and so resemble the splittable fibre prior to the processing. However, another section of the splittable fibre will be split and so the smaller constituent fibres making up the splittable fibre may have separated to some extent. Alternatively, the entire length of the splittable fibre may be split into its constituent fibres.

A splittable fibre may include at least one of each of the constituent fibres. For example, if a splittable fibre contains only one of each of the constituent fibres, then the splitting results in the two constituent fibres being separated from each other along at least part of their length. As another example, if a splittable fibre contains at least two of each of the constituent fibres, then the splitting may be such that only one of one of the constituent fibres is separated from the other constituent fibre. That is, for a splittable fibre to be considered split, it is adequate that only a single constituent fibre has been separated in the splittable fibre.

As an example, a bicomponent splittable fibre may be produced by a co-extrusion process to form two different types of fibres—for example, two different polymer fibres—alongside each other. However it will be appreciated that other methods of producing a splittable fibre may exist. A bicomponent fibre may therefore include at least one fibre of each of the two types of fibres. In some examples, a polymer fibre may be a fibre of PET, PL, Nylon, PP or PLA. For example, a bicomponent splittable fibre may include fibres of PET and fibres of Nylon. In certain embodiments, a bicomponent fibre may consist of a singular or multiple polymers.

While the whole splittable fibre itself may not be considered to be a microfibre according to the above definition, the constituent fibres of the splittable fibre may be considered a microfibre according to this definition. Accordingly, the process of splitting the splittable fibre may be considered to result in microfibres. That is, upon splitting, at least one of the now-separated constituent fibres of a splittable fibre may have a linear density approximately equal to or less than 1 dtex.

In certain embodiments, the wound products obtainable by performing the methods disclosed herein exhibit splittable fibres having a linear density of less than 1 dtex. In some embodiments the wound products obtainable by performing the methods disclosed herein comprise fibres having a linear density of less than 1 dtex and fibres having a linear density greater than 1 dtex. For example, the wound products may comprise fibres having a linear density of less than 1 dtex and fibres of between 1 dtex and 15 dtex.

In certain embodiments, the constituent fibres of a splittable fibre may be chosen according to the desired characteristics of the processed, or pre-processed, splittable fibre. It will be appreciated that the present invention is not limited to any particular combinations of constituent fibres.

In certain embodiments, a splittable fibre (or at least some of the splittable fibres in the first layer) may include a spin finish or processing agent. That is, a splittable fibre may be coated in a spin finish or a processing agent, wherein the spin finish or processing agent is adapted to aid processing. In certain embodiments, a low-level water soluble spin finish is added to at least some of the splittable fibres in the first layer.

In yet other embodiments, an active agent may be added to some of the splittable fibres. For example, silver may be added to the splittable fibres to help improve antimicrobial properties of the wound product. As another example, iodine may be added to the splittable fibres. In another example, an antimicrobial additive including one or more of elemental silver, silver salts, silver complexes, caged forms of silver, and caged iodine and combinations thereof may be added to the splittable fibres. In some examples, silver complexes and silver salts are selected from one or more of colloidal silver, silver zeolite, silver sulfadiazine, silver sulfate, silver carbonate, silver chloride, silver nitrate, silver oxide, silver phosphate, silver citrate, silver acetate, silver lactate, and combinations thereof. Furthermore, in some examples, caged iodine is selected from cadexomer iodine.

In certain embodiments of the present invention, the first layer may also include at least one additional fibre or type of fibres. For example, in addition to the plurality of splittable fibres, the first layer may include viscose fibres or synthetic fibres (such as PET). Accordingly, the first layer may be regarded as a blend of splittable fibres and such additional fibres. As an example, a first layer including such a blend of fibres may made up of 15% to 90% splittable fibres, with the remainder being viscose fibres or synthetic fibres—some exemplary fibre blends are discussed below.

In an example, viscose fibres may be included in the first layer to add strength and improve processing of the first layer. In another example, thermoplastic fibres, or non-splittable bicomponent fibres, may be added to the first layer and a bonding procedure (for example, employing air oven or calendaring methods) performed to provide further bonding within the first layer and increase strength. As will be described later, in certain embodiments the processing to split at least some of the splittable fibres in the first layer may itself be said to result in bonding (mechanical entanglement) of at least some split splittable fibres, and so the bonding procedure when thermoplastic fibres, or non-splittable bicomponent fibres, are added to the first layer may be considered to be in addition to any bonding which occurs during the processing.

For example, in addition to the splittable fibres, the first layer may include at least some other fibres (or other fibre types), which may be one or more of: PET, PP, PLA, Viscose, Cellulose, PES and PE. Here, it may be said that the first layer includes a fibre blend comprising at least some splittable fibres and at least some of one or more of these other fibres (other fibre types). Furthermore, the ratio of the different types of fibres included in the first layer may be adjusted according to the desired properties of the first layer and the resulting wound product. Some examples of ratios for different fibres in some exemplary fibre blends which may be included in a first layer are as follows: 30% splittable fibres, 50% viscose, 20% PET; 50% splittable fibres, 25% viscose, 25% PET; 30% splittable fibres, 30% viscose, 30% PET, 10% other bicomponent fibres (here, an additional bonding step may be employed, as mentioned in the previous paragraph); 60% splittable fibres, 40% viscose; and 80% splittable fibres, 20% PET. Of course, it will be appreciated that other ratios and fibre blends are possible and these are simply non-binding, illustrative examples.

Examples of splittable fibres which may be used in certain embodiments of the present invention are shown in FIGS. 2A to 2E. It will be appreciated that the types of splittable fibres shown in FIGS. 2A to 2E are merely exemplary and should not be considered limiting on the present invention, with other types of splittable fibres also being suitable for use in the present invention.

FIG. 2A illustrates a splittable fibre 222 which is a splittable fibre having a “pie” structure. As can be seen, such a splittable fibre includes wedges of a first constituent fibre 2221, the first constituent fibre 2221 being of a first type of constituent fibre, and wedges of a second constituent fibre 2223, the second constituent fibre 2223 being of a second type of constituent fibre, which are arranged in a substantially circular shape.

FIGS. 2B to 2D illustrate splittable fibres having an “island(s)” structure or “sea” structure. As can be seen, such a splittable fibre may include a first constituent fibre as a sheath portion, the first constituent fibre being of a first type of constituent fibre, and at least one of second constituent fibres which are at least partly surrounded or encased by the first constituent fibres and extend longitudinally through the first constituent fibre, the second constituent fibres being of a second type of constituent fibre.

In FIG. 2B, splittable fibre 224 may be generally referred to as an islands structure, wherein splittable fibre 224 includes a first constituent fibre 2241 and a plurality of second constituent fibres 2243 within the first constituent fibre 2241.

As a further example of such an islands structure, FIG. 2C illustrates splittable fibre 226 which includes a first constituent fibre 2261 and three second constituent fibres 2263 within the first constituent fibre 2261.

FIG. 2D shows an island type splittable fibre 228 which includes a first constituent fibre 2281 and only a single second constituent fibre 2283 within the first constituent fibre 2281. This splittable fibre 228 may be referred to as having a “sheath/core” structure or, if the single second constituent fibre is offset from the centre of the first constituent fibre, having an “eccentric sheath/core” structure.

Where splittable fibres 222, 224, 226, 228 of FIGS. 2A to 2D have a substantially round cross-section, it will be appreciated that the splittable fibre may also have a modified cross-section. An example of this is shown by splittable fibre 230 in FIG. 2E. Here, a trilobal cross-section for a splittable fibre is shown, specifically a “tipped trilobal” splittable fibre where the tips 2301 are a different constituent fibre to the central part 2303. That is, the tips are formed of a different material to the central part, and upon the splittable fibre splitting longitudinally along at least part of its length at least one of the tips is separated from the central part along that part of the length of the split splittable fibre.

Modified cross-sections may allow for additional functionality in the sub-fibres resulting from the splitting of a splittable fibre and/or the splittable fibres themselves, for example allowing a degree of control over lustre or moisture transport. The present invention should not be considered limited to the cross-sections illustrated in FIGS. 2A to 2E, and indeed a wide variety of fibre cross-sections are suitable for use with the present invention.

In step 120 of FIG. 1, the first layer is processed such that at least some of the plurality of splittable fibres are split longitudinally along at least part of their length. Further, the processing results in mechanical entanglement of the splittable fibres.

For example, the processing may result in: only a single splittable fibre included in the first layer being split along at least part of its length; a plurality of splittable fibres included in the first layer being split along at least part of their length, where such splitting may be different (that is, the length of the splittable fibre which is split may be different) for each splittable fibre which is split; or all of the splittable fibres included in the first layer may be split, and again such splitting may be different for each splittable fibre.

As mentioned previously, the split part(s) of a splittable fibre (that is, the now-separated constituent fibres) may be termed sub-fibre(s), and at least some of the sub-fibre(s) may meet the definition of a microfibre given above.

In certain embodiments of the present invention, the processing includes hydroentangling (or spunlacing). That is, the first layer is processed into a nonwoven fabric through a hydroentanglement process. Accordingly, certain embodiments of the present invention produce a wound product which includes a plurality of microfibres, wherein the microfibres result from hydroentangling a layer of splittable fibres.

In hydroentangling, the first layer including the plurality of splittable fibres is passed through a one or more jets of liquid. The jets of liquid may be high pressure water jets, for example. The pressure of the liquid jets may change during the processing. The pressure of individual liquid jets may also differ from one another. For example, a first liquid jet that the first layer is passed through in a predetermined direction may be at a lower pressure than the last liquid jet that the first layer is passed through in the predetermined direction. According to certain embodiments, hydroentanglement pressure (the pressure of the liquid jets) may range from approximately 30 bar to approximately 200 bar, where the pressure used may depend on fibre type, the weight of the splittable fibre web (the first layer prior to hydroentangling), the intended end use of the fibre web (for example, this may be related to the desired properties of a wound product produced by the method, where it would be appreciated how the hydroentanglement pressure may affect the degree of entanglement between the fibres included in the first layer), etc.

The jets of liquid may be regarded as being used for splitting and needle punching the splittable fibres in the first layer. That is, as the jets of liquid contact the first layer and so at least some of the splittable fibres, at least some of the contacted splittable fibres are split, along at least part of their length, into a plurality of fibre segments (being the constituent fibres of the splittable fibre). Furthermore, as the jets of liquid contact the first layer, at least some of the splittable fibres are bonded together. Such bonding may be by mechanical entanglement between the splittable fibres. Furthermore, the sub-fibres split of the split splittable fibres may also be bonded. Such bonding may be to other sub-fibres or to splittable fibres, and may also be by mechanical entanglement.

Upon having undergone the processing, for example the spunlacing, a wound product according to certain embodiments of the present invention has been manufactured. In certain embodiments, the method may include a further step whereby excess liquid from the hydroentangling is removed from the first layer, or wound product. This may be achieved, for example, by using a dewatering system. Furthermore, in certain embodiments as will be described in greater detail below, the method may further include bonding the first layer to an additional layer to produce a wound product having additional characteristics.

FIG. 3 illustrates an example of how a splittable fibre may appear before and after splitting (or having been processed). In this example, the splittable fibre 310a has the pie structure shown in FIG. 2A. Prior to processing, wedges of the first constituent fibre 312 and wedges of the second constituent fibre 314 are arranged in a substantially circular pattern in splittable fibre 310a, where the wedges alternate between the first and second constituent fibres.

After processing, the processed splittable fibre 310b shows separation between at least some of the first and second constituent fibres. Specifically, in this example there is a large degree of separation shown between the first constituent fibres 312 and the second constituent fibres 314. However, referring to the above discussion, if only a single one of the illustrated wedges was shown to be separated, the processed splittable fibre 310b may still be considered to have been split longitudinally along at least part of its length.

According to certain embodiments of the present invention, a wound product including a first layer as described above may include at least one additional layer. In one example, such an additional layer may be arranged on a wound facing side of the first layer such that, when the wound product is applied to a wound, the first layer is furthest from the wound. That is, the additional layer may be a wound contact layer and would be substantially between the wound and the first layer. The wound contact layer may be a permeable film layer or silicon adhesive layer, for example. In another example, such an additional layer may be enclosed within the first layer. That is, the splittable fibre layer including some split fibres and mechanically entangled splittable fibres may encase at least one additional layer or at least one additional material. Accordingly, in such embodiments, the method may further include bonding such an additional layer to the first layer, or providing such an additional layer in the wound product in addition to the first layer.

In one implementation, the first layer acts as a scrim layer for an additional layer. That is, the wound product includes the first layer as a splittable fibre scrim layer. In certain embodiments of the present invention, such a splittable fibre scrim layer may then be bonded to, or have bonded to it, a layer of superabsorbent fibres or gelling fibres. Such bonding may be achieved, for example, through needlepunching, air-oven bonding, calendaring, or chemical bonding. For example, a superabsorbent layer may be laminated with an adhesive and then applied to the processed first layer in which at least some of the splittable fibres are split longitudinally along at least part of their length and are mechanically entangled. In another example, the superabsorbent or gelling material may be provided initially in a powdered form which may then be applied to the processed first layer to form a layer of superabsorbent or gelling material. The first layer is aptly completely dried after the hydroentanglement process prior to the application of the superabsorbent layer. This may help to prevent swelling of the superabsorbent material prior to use on a wound.

As described above, the wound product of certain embodiments may be considered as a nonwoven fabric according to the processing method used for splitting and mechanically entangling the first layer. It will be appreciated that, in general, such a nonwoven fabric may be laminated to further nonwoven fabrics or layers, foams, films or wound contact layers.

For example, in certain embodiments, the wound product manufactured by the method shown in FIG. 1 may also include a layer of superabsorbent fibres or gelling fibres. In another example, the first layer of the wound product may encase loose superabsorbent or gelling fibres or particles.

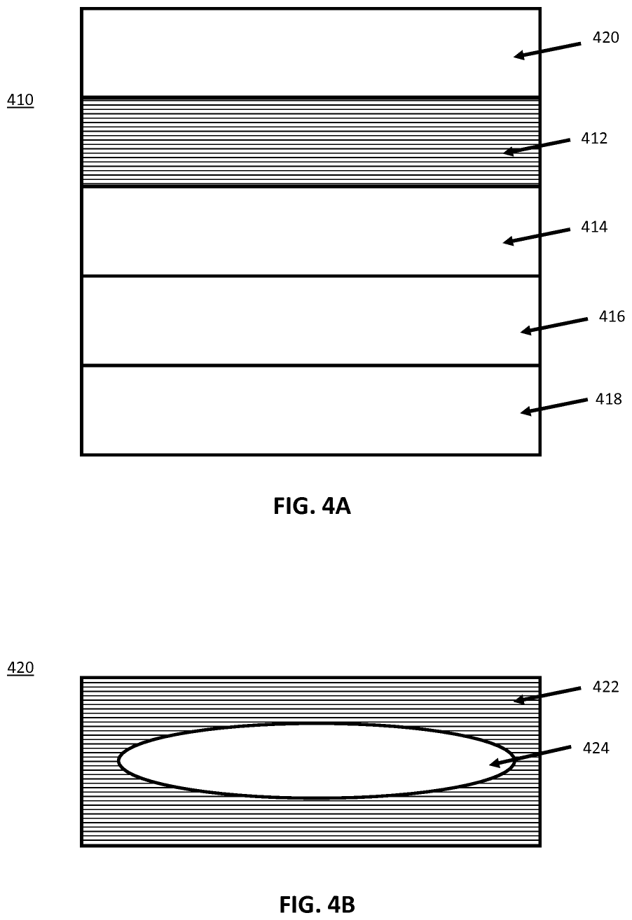

FIG. 4A illustrates a wound product 410 in accordance with certain embodiments of the present invention. As described above, the wound product 410 may be a wound dressing or a wound filler. In other embodiments, the wound product 410 may be a debrider or a wound cleaner. These are merely exemplary though, and should not be considered as limitations to a wound product in accordance with the present invention.

In FIG. 4A, a wound product 410 in accordance with an embodiment of the present invention is illustrated. The wound product 410 includes a first layer 412 including a plurality of splittable fibres. The first layer 412 has been processed, for example by hydroentangling (spunlacing), such that at least a subset of the splittable fibres are split longitudinally along at least part of their length, and wherein the processing has resulted in mechanical entanglement of the splittable fibres or at least some of the splittable fibres. In an example, the splittable fibres of the first layer 412 may be bicomponent fibres as described above, such fibres including first and second constituent fibres at least some of which are separated, to an extent, due to the processing. Additionally, as described above, the splittable fibres may have optionally been coated with a spin finish, and/or an active agent may have been added to the splittable fibres (either prior to, during or after processing).

FIG. 4A also shows first, second, third and fourth additional layers 414, 416, 418, 420 in the wound product 410, however it will be appreciated from the above that these additional layers 414, 416, 418, 420 are optional modifications to wound product 410.

First additional layer 414 may be a layer of superabsorbent fibres or gelling fibres as described above. For example, upon forming and processing the first layer 412 in accordance with one of the examples described above, the first layer 412 may be laminated to the first additional layer 414. Alternatively, in another example, the first layer 412 may be used as a scrim to which the first additional layer including a superabsorbent or gelling fibre layer is bonded to by needlepunching. In such an example, using the first layer, which includes at least some split fibres (which, as described above, may be microfibres), as a scrim may provide improved absorbency compared to a meltspun nonwoven scrim by providing an increased surface area. In another example, other materials which may be used in such a laminate construction may be a knitted spacer fabric, extruded nets or films.

In certain embodiments, the wound product 410 may include a second additional layer 416. In one example, the second additional layer 416 may be a foam layer, for example an open cell PU foam. A foam layer may provide the advantage of helping to transport exudate away from the wound. Here, the second additional layer 416 may be arranged such that the first additional layer 414 is between the second additional layer 416 and the first layer 412, such that the second additional layer 414 would be more proximate to a wound than the first additional layer 416. Of course, it will be appreciated that the arrangement of layers in this example is not limiting, and they may be arranged in the reverse order to that described above.

In certain embodiments, a third additional layer 418 may be a wound contact layer. An example of a wound contact layer is a layer including silicon adhesive or perforated film.

In certain embodiments, this third additional layer 418, being a wound contact layer, may be present in a wound product 410 along with one or more of a first additional layer 414 (for example, a superabsorbent or gelling fibre layer) and a second additional layer (for example, an open cell PU foam). The third additional layer 418 may be arranged such that the third additional layer 418 would be more proximate to a wound that the first and second additional layers 414, 416. For example, a third additional layer 418 may be a wound contact layer arranged such that the third additional layer 418 would be substantially between the wound and the other layers of the wound product 410. Of course, it will be appreciated that the arrangement of layers in this example is not limiting.

In certain embodiments, a fourth additional layer 420 may be included in the wound product 410, as shown in FIG. 4A. The fourth additional layer 420 may be, for example, a moisture vapour permeable/liquid impermeable layer.

In some examples, the fourth additional layer 420 may be included in the wound product 410 in addition to one or more other additional layer 414, 416, 418. For example, a wound product 410 according to an embodiment of the present invention may include a first layer 412, a first additional layer 414 (which may be a layer of superabsorbent or gelling fibres, for example), a second additional layer 416 (which may be a foam layer, for example), a third additional layer (which may be a wound contact layer, for example) and a fourth additional layer 420 (which may be a moisture vapour permeable/liquid impermeable layer, for example). The fourth additional layer 420 may be arranged on a side of the first layer 412 opposite to the side on which at least one of the first, second and third additional layers 414, 416, 418 is arranged. For example, if first, second and third additional layers 414, 416, 418 are arranged on a wound facing side of the first layer 412, the fourth additional layer 420 may be arranged on the other side (the non wound facing side) of the first layer 412. Of course, it will be appreciated that the arrangement of layers in this example is not limiting.

In certain embodiments, the second additional layer 416 may be bonded to the first layer 412 around the periphery (for example, by an adhesive layer (not shown)), so that a first additional layer 414 is sandwiched between the first layer 412 and the second additional layer 416. In other embodiments, the third additional layer 418 may be bonded to the first layer 412 around the periphery (for example, by an adhesive layer (not shown)), so that a first additional layer 414 and a second additional layer 418 are sandwiched between the first layer 412 and the third additional layer 418. It will be appreciated that, in other examples, such bonding may be effected between other layers included in a wound product according to an embodiment of the present invention. For instance, two additional layers may be bonded at their respective peripheries, sandwiching in between another additional layer and/or the first layer 412.

It will be appreciated that, although not shown in FIG. 4A, further additional layers may be provided on the side of the wound product 410 other than the side on which the first, second and third additional layers 414, 416, 418 have been shown to be provided. For example, another additional layer could be provided on the side of the wound product on which the fourth additional layer 420 is located, either between the first layer 412 and the fourth additional layer 420 or such that the fourth additional layer 420 is between the first layer 412 and the another additional layer. For example, a seal layer (included for a wound product which may be used in a negative pressure wound therapy system) could be provided on the side of the fourth additional layer 420 where the first layer 412 is not located.

FIG. 4B schematically illustrates another example of a wound product 420. Here, the wound product 420 includes a first layer 422 and a first additional layer 424 which is encased, at least partly, by the first layer 422. The first layer 422 may be a layer of processed splittable fibres in accordance with one of the examples described above. The first additional layer 424 may be a superabsorbent or gelling fibre layer in accordance with one of the examples described above. The first additional layer may be superabsorbent or gelling particles, or superabsorbent or gelling loose fibres, for example. Although not shown, it will be appreciated that further additional layers may be present. For example, the wound product 420 shown in FIG. 4B may include, on one side of the first layer 422, a foam layer (for example, an open cell PU foam). Similarly, in other examples, one or more other additional layers such as a wound contact layer and a moisture vapour permeable/liquid impermeable layer may be included.

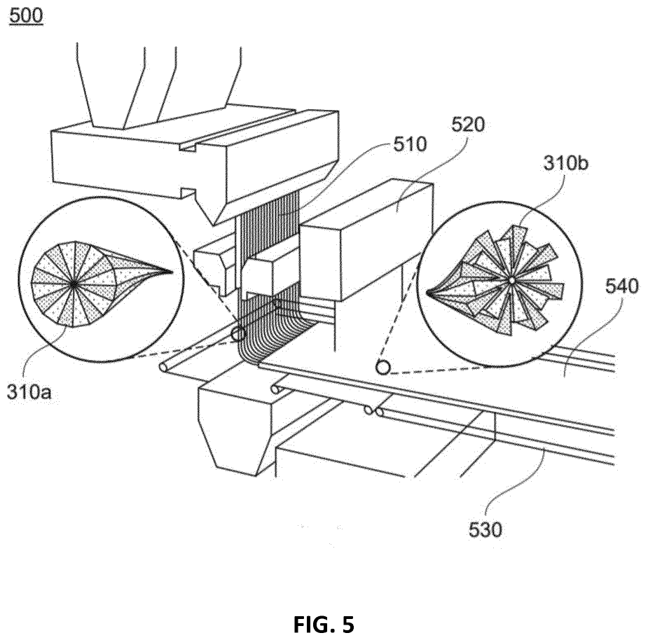

FIG. 5 illustrates an exemplary apparatus 500 for performing a method of manufacturing a wound product according to certain embodiments of the present invention.

A layer 510 including a plurality of splittable fibre 310a is introduced or fed into the apparatus. This layer 510 may be in accordance with any of the examples described above. For example, the layer 510 may include a plurality of bicomponent splittable fibres which have, or have not, had a spin finish applied. The layer 510 may therefore be considered as a first layer as described above but prior to the processing which results in splitting and entangling.

The layer 510 may then be supported by a surface 530, such as a drum or belt. While supported on the surface 530, the layer 510 is passed through jets of liquid 520 (for example, high pressure water jets) which contact the layer 510 and cause splitting of at least some of the splittable fibres 310a in the layer 510. The processed layer 540 which has passed through the jets of liquid 520 therefore includes at least some split fibres 310b. Accordingly, the processed layer 540 may be considered similar to the first layer 412 which has been processed and so includes at least some split splittable fibres with mechanical entanglement of the splittable fibres, as described above in relation to various embodiments of the present invention.

In certain embodiments, the apparatus 500 may include several components 520 which provide jets of liquid. Alternatively or additionally, the apparatus 500 may be configured to pass the layer 510 through a jet of liquid in a first direction and then pass the layer 510 through the same jet of liquid in a second direction which may be opposite to the first direction, thereby allowing for a reduction in a number of components providing jets of liquid, or alternatively may help to increase the proportion of split fibres.

Although not shown, it will be appreciated how the apparatus could be modified such that the processed layer 540 is then passed through further machinery which results in bonding the processed layer 540 to at least one additional layer 414, 416 such as described above. However, in such modified apparatus, it may be desirable to dry the processed layer 540 before any such bonding. For example, if the layer 510 is subjected to hydroentangling using jets of water, attempting to bond the still-wet processed layer 540 to a superabsorbent layer may detrimentally affect the superabsorbent layer and so result in a subpar wound product. Accordingly, suitable drying means may be provided in such a modified apparatus.

Various embodiments have been described above wherein the method of manufacturing the wound product comprises processing the splittable fibres in such a manner that said processing step results in splitting the fibres longitudinally along at least part of their length and also enables mechanical entanglement of the splittable fibres. Advantageously, both splitting and entanglement can occur during a single operation. This may be achieved by the use of, for example, a hydroentanglement process such as that outlined above.

In some alternative embodiments of the method described herein the splitting and mechanical entanglement of the splittable fibres may be achieved by performing separate, discrete operations. After forming a first layer comprising a plurality of splittable fibres, the next steps in the method may comprise (i) performing a process to split the fibres longitudinally along at least part of their length and then, subsequently, (ii) performing a further process that results in mechanical entanglement of the splittable fibres.

In yet a further embodiment, the aforementioned process can be re-ordered such that after forming a first layer comprising a plurality of splittable fibres, the next steps in the method comprise (i) performing a process that results in mechanical entanglement of the splittable fibres and then, subsequently, (ii) performing a process to split the fibres longitudinally along at least part of their length.

Where separate operations are used to achieve splitting and mechanical entanglement of the splittable fibres, different methods known in the art may be employed. For example, splitting the fibres can be achieved via the application of pressure such as in a milling process. Pressure may be applied via rollers, such as in a nip process or via continuous or discontinuous application of pressure such as a stamping or punching process. Additionally, mechanical entanglement of the fibres can result from the use of needlepunching. Fibre bonding may also be achieved via thermal or chemical bonding.

Any of the examples described herein may be adapted for use with a negative pressure system (sometimes referred to as a reduced pressure system) including a source of negative pressure, such as a negative pressure pump. For example, a wound product may include a negative pressure interface, such as a port, to which a negative pressure supply tube may be connected. For example, for certain wound dressing embodiments of a wound product, a seal layer may be included on a non wound facing side of the first layer, wherein the seal layer includes the negative pressure interface. The seal layer may be selected to be such that, when the wound dressing is applied to a wound, the seal layer will aid in maintaining negative pressure in the wound. As another example, for certain wound filler embodiments of a wound product, the wound filler may be placed inside a wound cavity, and a cover (for example, a seal layer) may be placed over the wound cavity to maintain negative pressure in the wound, where the cover may include a negative pressure interface to which a negative pressure supply tube may be connected. In some embodiments, the cover may comprise a moisture vapour permeable film. Of course, the person skilled in the art would not consider these methods of using a wound product in a negative pressure system as being limiting, and that other methods are available.

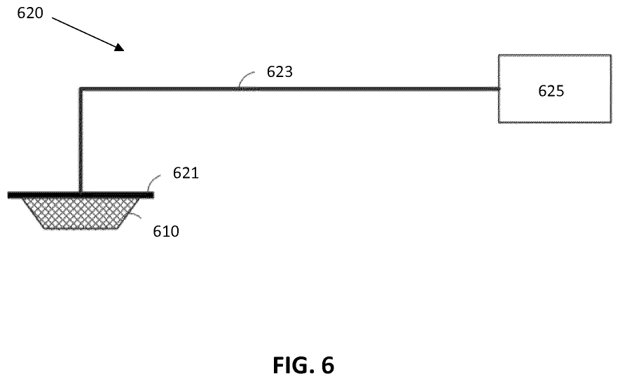

FIG. 6 illustrates an example of a negative pressure wound therapy system 620, in which the wound product of the present invention is embodied as a wound dressing. FIG. 6 illustrates a wound cavity 610 covered by a wound dressing 621, which can be a wound dressing according to any of the examples of a wound product described herein. The wound dressing 621 can be positioned inside the wound cavity 610 and further seal the wound cavity so that negative pressure can be maintained in the wound cavity. For example, a seal layer of the wound dressing 621 can provide substantially fluid impermeable seal over the wound cavity 610. A single or multi lumen tube or conduit 623 connects the wound dressing 621 with a negative pressure device 625 configured to supply reduced pressure. The negative pressure device 625 includes a negative pressure source. The negative pressure device 625 can be a canisterless device (meaning that exudate is collected in the wound dressing and/or is transferred via the tube 623 for collection to another location). In some embodiments, the negative pressure device 625 can be configured to include or support a canister. Additionally, in any of the embodiments disclosed herein, the negative pressure device 625 can be fully or partially embedded in, mounted to, or supported by the wound dressing 621.

The conduit 623 can be any suitable article configured to provide at least a substantially sealed fluid flow path or pathway between the negative pressure device 625 and the wound cavity 610 so as to supply reduced pressure to the wound cavity. The conduit 623 can be formed from polyurethane, PVC, nylon, polyethylene, silicone, or any other suitable rigid or flexible material. In some embodiments, the wound dressing 621 can have a port configured to receive an end of the conduit 623. For example, a port can include a hole in the seal layer. In some embodiments, the conduit 623 can otherwise pass through and/or under a seal layer of the wound dressing 621 to supply reduced pressure to the wound cavity 610 so as to maintain a desired level of reduced pressure in the wound cavity. In some embodiments, at least a part of the conduit 623 is integral with or attached to the wound dressing 621.

In certain embodiments of the present disclosure, the wound product may be suitable to include within a negative pressure wound apparatus. In certain embodiments, the wound product may be a negative pressure wound dressing.

In certain embodiments, the wound product may be used as the dressing component of a negative pressure wound dressing apparatus. The apparatus in different embodiments comprises a canister and is free of the canister.

In one embodiment, there is provided a negative pressure wound therapy kit comprising a wound product as described above and a negative pressure source configured to be fluidically connected to the wound product.

In one embodiment, there is provided a method of providing negative pressure wound therapy to a wound, the method comprising: placing a wound product such as described above over a wound; forming a fluid flow path between the wound dressing and a negative pressure source; and operating the negative pressure source to provide negative pressure to the wound.

In one embodiment, there is provided a method of operating a negative pressure wound system, the method comprising: operating a negative pressure source fluidically connected to a wound product such as described above, the wound product configured to be positioned over a wound.

Wound products in accordance with certain embodiments of the present invention may provide an advantage of greater absorbency over prior art nonwoven wound products including fibres of >1 dtex. For a suitably prepared wound product, total absorbency and liquid wicking rate may be measured according to ISO 9073-6:2000, test method for nonwovens, Absorption. This method can measure amount of liquid a textile can hold.

As may be appreciated, this may allow for the production of a wound product which, in comparison to prior art wound products, has equivalent absorbency but a smaller profile. For example, the processed first layer may be thinner than an equivalently-absorbent prior art nonwoven scrim.

Furthermore, wound products in accordance with certain embodiments of the present invention may provide an advantage of an improved drying rate. Drying rate may be measured according to AATCC TM200-2016, Drying rate of textiles at their absorbent capacity.

Furthermore, wound products in accordance with certain embodiments of the present invention may provide an advantage of improved water vapour transmission. This may follow from the spreading of liquid, within the wound product, over a much greater surface area, which may result in the absorbed liquid being evaporated off much quicker. Water vapour transmission rates may be measured using the Sweating Guarded Hot Plate Method (ISO 11092)

In some embodiments the wound products disclosed herein can be used as a wound dressing that forms a component of a negative pressure wound therapy apparatus or kit that does not include a canister for collecting exudate from the wound. The wound products may thus comprise part of a “canisterless” negative pressure wound therapy system intended to retain substantially all of the wound exudate within the wound dressing. To this end, the wound products may comprise superabsorbent material to enhance the absorbent capacity of the wound dressing. Furthermore, a cover layer in the form of a moisture vapour permeable top film may be utilized to promote evaporation of exudate from the dressing thereby further increasing its absorbent capacity. Accordingly, the use of the wound products disclosed herein is particularly advantageous in canisterless negative pressure wound therapy systems as the useable capacity of the wound dressing is enhanced by virtue of the improved fluid handling characteristics arising from the spreading of liquid and greater absorbancy within the wound product.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1.-34. (canceled)

35. A method of manufacturing a wound product, the method comprising:
forming a first layer comprising a plurality of splittable fibres; and
processing the first layer such that at least some of the plurality of splittable fibres are split longitudinally along at least part of their length.

36. The method of claim 35, wherein the processing results in mechanical entanglement of the splittable fibres.

37. The method of claim 35, further comprising subsequently performing a process that results in mechanical entanglement of the splittable fibres.

38. The method of claim 35, wherein the wound product is one of a wound dressing and a wound filler.

39. The method of claim 35, wherein the wound product comprises fibres having a linear density <1 decitex (dtex).

40. The method of claim 35, wherein the wound product comprises fibres having a linear density <1 decitex (dtex) and fibres having a linear density of between 1 decitex (dtex) and decitex (dtex).

41. The method of claim 35, wherein a splittable fibre of the plurality of splittable fibres comprises at least two constituent fibres; and
wherein, in a splittable fibre split longitudinally along at least part of its length, a first constituent fibre is separated from a second constituent fibre for the part of the length of the split splittable fibre.

42. The method of claim 41, wherein at least one of the first constituent fibre and the second constituent fibre are fibres having a linear density <1 decitex (dtex).

43. The method of claim 35, wherein processing the first layer comprises hydroentangling the first layer.

44. The method of claim 35, further comprising bonding the first layer to a second layer, wherein the second layer comprises at least one of a plurality of superabsorbent fibres and a plurality of gelling fibres.

45. The method of claim 44, wherein bonding the first layer to the second layer comprises needlepunching the second layer to the first layer, wherein the first layer is provided as a scrim.

46. The method of claim 44, wherein bonding the first layer to the second layer comprises laminating at least one of the first layer and the second layer with an adhesive.

47. The method of claim 44, wherein bonding the first layer to the second layer comprises encasing the second layer within the first layer.

48. The method of claim 35, further comprising encasing a second layer within the first layer, wherein the second layer comprises at least one of loose superabsorbent fibres or particles and loose gelling fibres or particles.

49. The method of claim 35, wherein the method further comprises bonding the first layer to at least one additional layer; and
wherein an additional layer is one of a superabsorbent or gelling fibre layer, a foam layer, a wound contact layer and a moisture vapour permeable/liquid impermeable layer.

50. The method of claim 35, wherein forming the first layer comprises one or more of: applying a spin finish to at least some of the splittable fibres, and applying silver particles to at least some of the splittable fibres.

51. The method of claim 35, wherein at least some of the splittable fibres have a pie structure, an island structure, a trilobal cross-section, or a round cross-section.

52. The method of claim 35, wherein the first layer comprises at least some other fibres;
wherein the other fibres are at least one of: PET, PP, PLA, Viscose, Cellulose, PES and PE fibres.

53. The method of claim 52, wherein the splittable fibres form 15% to 90% of the first layer.

54. A method of manufacturing a wound product, the method comprising:
forming a first layer comprising a plurality of splittable fibres;
performing a process that results in mechanical entanglement of the splittable fibres; and
subsequently processing the first layer such that at least some of the plurality of splittable fibres are split longitudinally along at least part of their length.
